(19) United States
(12) Patent Application Publication
(10) Pub. No.: US 2025/0246321 A1
(43) Pub. Date: Jul. 31, 2025

(54) Devices, Systems, and Methods for Generating and Providing Personalized Communications to Improve Adherence to Patient Treatment Plans

(71) Applicant: Elevance Health, Inc., Indianapolis, IN (US)

(72) Inventors: Ayush Mathur, Novi, MI (US); Judy Fujimoto, Manhattan Beach, CA (US); Anthony Nguyen, Pacific Palisades, CA (US); Caroline Bostwick Spencer, Greencastle, IN (US); Vinay Kumar Boddula, Atlanta, GA (US); Umesh C. Rath, Suwanee, GA (US)

(21) Appl. No.: 19/040,827

(22) Filed: Jan. 29, 2025

Related U.S. Application Data
(60) Provisional application No. 63/626,890, filed on Jan. 30, 2024.

(51) Int. Cl. G16H 80/00; G06Q 50/00; G16H 10/60; G16H 20/10; G16H 50/30

(52) U.S. Cl. CPC G16H 80/00; G06Q 50/01; G16H 10/60; G16H 20/10; G16H 50/30

(57) ABSTRACT

The present disclosure regards an electronic device configured to generate personalized communications regarding patient treatment plans. The electronic device includes a processor configured to perform various operations. These operations include receiving data regarding a patient, where the data includes treatment plan data regarding a treatment plan prescribed for the patient and other data regarding a healthcare provider, a clinician, a medical record, a social media account, a laboratory, or a pharmacy associated with the patient. The operations also include providing the data to a plurality of machine learning (ML) models configured to generate a personalized communication for the patient based on the data. Additionally, the operations include receiving the personalized from the plurality of ML models, where the personalized communication is addressed to the patient and regards the treatment plan prescribed for the patient. Further, the operations include delivering the personalized communication to the patient.

### CROSS-REFERENCE TO RELATED APPLICATIONS

This application claims the benefit of U.S. Provisional Application No. 63/626,890 filed Jan. 30 2024, the entirety of which is incorporated herein by reference.

### TECHNICAL FIELD

The present disclosure relates, generally, to machine learning (ML) and, more particularly, to ML models configured to generate personalized communications regarding patient treatment plans.

### BACKGROUND

Treatment adherence refers to the degree to which people follow treatments prescribed by their physicians. It is key in achieving optimal health outcomes, especially for people dealing with chronic diseases. And yet, studies suggest that treatment adherence is exceptionally low. Some studies, for example, indicate that treatment adherence is as low as 50% amongst people suffering from chronic illnesses.

Several factors may be to blame for treatment nonadherence. The cost of prescribed treatments presents a significant hurdle for patients with limited financial resources. Additionally, treatment complexity often results in patients struggling to correctly follow prescribed instructions or ignoring them altogether. Moreover, a lack of understanding can result in nonadherence, as patients fail to grasp the importance or the potential benefits of treatments prescribed for them.

The consequences of nonadherence are often serious and can lead to hospitalization, disability, and even death. The burden of nonadherence is borne also by patients' families, their physicians, and the general public. Symptoms that might otherwise have been avoided can lead to increased healthcare costs, increased workloads for physicians, and increased stress for family members.

In short, treatment adherence benefits everyone in the healthcare industry, from patients and their families to physicians and healthcare providers. Nonetheless, many patients still struggle or choose not to stick to their prescribed treatments. There is therefore a pressing need for innovative technologies to improve adherence by addressing the challenges noted above.

### SUMMARY

Adherence to prescribed treatments has historically not improved with delivery of treatment requirements to patients by providers and payors. Factors affecting adherence include patient motivation and treatment plan complexity (e.g., including unclear or confusing instructions). Unfortunately, many healthcare providers are not trained in the motivational delivery of treatment plan instructions, nor do they have the capacity to properly follow up and push for initial and continued adherence.

The innovations discussed herein seek to improve treatment plan adherence by utilizing ML and other technologies (e.g., electronic medical records) to craft and deliver personalized, motivational communications for patients regarding their treatment plans. Through large-language modeling technologies, a personalized communication can be tailored to account for various aspects of the treatment plan, such as its complexity and the difficulty of adhering to it. The personalized communication can also be adjusted based on the patient to which it is addressed, accounting for factors such as the patient's communication style and the patient's understanding of their illness or disease.

Exemplary embodiments of the subject technology include the following:

A system for improving adherence to patient treatment plans includes a transformer, a storage layer, a plurality of ML models, a delivery network, and an electronic device. The transformer is configured to vectorize data regarding a patient. The storage layer is configured to store the data. The plurality of ML models is configured to generate a personalized communication for the patient based on the aforenoted data. It includes a plan-complexity ML model trained using, for example, historical adherence data (e.g., for treatment plans similar to a treatment plan prescribed for the patient); a patient-persona ML model trained using, for example, unique interaction data and/or behavior pattern data (e.g., associated with patients similar to the patient); an adherence-probability ML model trained using, for example, a combination of persona data and historical adherence data; and a personalized-communication ML model trained using, for example, interaction data comprising language from interactions involving other patients (e.g., other patients similar to the patient). The delivery network is configured to deliver the personalized communication to the patient. The electronic device includes a processor configured perform operations. The operations include receiving the data regarding the patient, where the data includes treatment plan data regarding a treatment plan prescribed for the patient, and other data regarding a healthcare provider, a clinician, a medical record, a social media account, a laboratory, or a pharmacy associated with the patient. The operations also include vectorizing the data using the transformer, where vectorizing the data comprises translating the treatment plan data and the other data into numerical representations that capture semantic meaning embedded within the treatment plan data and the other data. Additionally, the operations include storing the data in the storage layer after vectorizing the data. Further, the operations include providing the treatment plan data from the storage layer to the plan-complexity ML model, where the treatment plan data includes a number of medications prescribed in the treatment plan, and a dosage frequency for the prescribed medications. Additionally, the operations include receiving a plan-complexity score from the plan-complexity ML model responsive to providing the treatment plan data to the plan-complexity ML model, where the plan-complexity score indicates a complexity level of the treatment plan. Further, the operations include providing the other data from the storage layer to the patient-persona ML model, where the other data regards at least the medical record associated with the patient and the social media account associated with the patient. Moreover, the operations include receiving persona indicators from the patient-persona ML model responsive to providing the other data to the patient-persona ML model, where the persona indicators indicate an ability of the patient to follow a prescribed regime and a communication style of the patient. Furthermore, the operations include providing the treatment plan data from the storage layer, the plan-complexity score, and the persona indicators to the adherence-probability ML model. Additionally, the operations include receiving an adherence-probability score from the adherence-probability ML model responsive to providing the treatment plan data, the plan-complexity score, and the persona indicators to the adherence-probability ML model, where the adherence-probability score indicates a probability of the patient adhering to the treatment plan. Further, the operations include providing the treatment plan data from the storage layer, the adherence-probability score, and the persona indicators to the personalized-communication ML model. Moreover, the operations include receiving a communication plan and the personalized communication from the personalized-communication ML model. The communication plan indicates whether the personalized communication should include text, images, or video and whether the personalized communication should be delivered via email, text message, notification, phone call, or letter. Also, the personalized communication is addressed to the patient and regards the treatment plan prescribed for the patient. Furthermore, the operations include delivering the personalized communication to the patient via the delivery network and according to the communication plan.

An electronic device configured to generate personalized communications regarding patient treatment plans includes a processor configured to perform operations. The operations include receiving data regarding a patient, where the data includes treatment plan data regarding a treatment plan prescribed for the patient and other data regarding a healthcare provider, a clinician, a medical record, a social media account, a laboratory, or a pharmacy associated with the patient. The operations also include providing the data to a plurality of ML models configured to generate a personalized communication for the patient based on the data. Additionally, the operations include receiving the personalized communication from the plurality of ML models responsive to providing the data to the plurality of ML models, where the personalized communication is addressed to the patient and regards the treatment plan prescribed for the patient. Further, the operations include delivering the personalized communication to the patient.

A computer-implemented method for improving adherence to patient treatment plans includes receiving data regarding a patient, where the data includes treatment plan data regarding a treatment plan prescribed for the patient, and other data regarding a healthcare provider, a clinician, a medical record, a social media account, a laboratory, or a pharmacy associated with the patient. The method also includes providing the data to a plurality of ML models configured to generate a personalized communication for the patient based on the data. Moreover, the method includes generating the personalized communication using the plurality of ML models after providing the data to the plurality of ML models, where the personalized communication is addressed to the patient and regards the treatment plan prescribed for the patient. Additionally, the method includes receiving the personalized communication from the plurality of ML models after generating the personalized communication. Further, the method includes delivering the personalized communication to the patient.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the figures and detailed description are to be regarded as illustrative in nature and not as restrictive.

### BRIEF DESCRIPTION OF THE FIGURES

For a better understanding of the various described embodiments, reference should be made to the Detailed Description below, in conjunction with the Figures. Like reference numerals refer to corresponding parts throughout the Figures and Description.

FIGS. 1A and 1B illustrate an exemplary system for generating personalized communications regarding patient treatment plans, according to various aspects of the subject technology.

FIG. 2 illustrates an exemplary process for improving adherence to a patient treatment plan, according to various aspects of the subject technology.

### DETAILED DESCRIPTION

As an example implementation of the technology discussed herein, consider the following: Patient is a middle-aged male who has been struggling with recent changes in his health and work status. He was recently diagnosed with diabetes and hypertension and passed over for a promotion at work. Patient sees these events as suggesting that he is “over the hill”; nonetheless, he is determined to regain control of his health and his career.

On his forty-ninth birthday, Patient set a goal to complete a marathon by age fifty. He purchased a fitness tracker to monitor his daily running distances, as well as his heart rate and sleep patterns. Patient was surprised that he was not able to hit his initial running targets due to mild wheezing after running for short distances. He logged this information in his fitness tracker and scheduled an appointment with his doctor for a check-up.

At a visit with his doctor, Patient learned that the doctor had also taken up running and recently completed a marathon. Patient and his doctor are about the same age, and Patient felt an instant connection. Patient mentioned to his doctor that he had not been taking his medications for diabetes and high blood pressure because he feels fine and often forgets to take them. He also admitted that the medications make him feel like a “sick person,” and he is determined to regain his health without medication. Additionally, Patient mentioned using an inhaler when exercising in the past but noted that he had not needed one for years—perhaps due to his sedentary lifestyle.

Patient's doctor offered Patient enrollment in a new service, where his doctor would provide ML-powered, personalized recommendations to help Patient achieve his goal of completing a marathon by age fifty. The recommendations would be customized based on Patient's personal goals and health conditions and would be optimized based on collating data submitted by Patient, as well as fitness tracker information, medical records, and other information. Patient's doctor also offered to add personal pointers from his own marathon training experience to the recommendations. Patient jumped at the opportunity and signed up for weekly reminders and advice from his doctor.

Within a week, Patient noticed a difference in his running and was able to hit his targets by using an inhaler prior to starting the run and taking his diabetes and high blood pressure medications as prescribed. He loves the personalized communications from his doctor, who was on a similar fitness journey not too long ago. Patient especially appreciates the reminders when he “forgets” to take his medication because he still resists the idea that he has a chronic disease. He is looking forward to his next check-up in three months, as he may be able to get off the medication. And if he forgets about the appointment, Patient knows that his doctor will be there to remind him.

Reference will now be made to embodiments, examples of which are illustrated in the accompanying figures. In the following description, specific details are set forth in order to provide an understanding of the various described embodiments. However, it will be apparent to one of ordinary skill in the art that the various described embodiments may be practiced without these specific details. In some instances, well-known methods, procedures, components, circuits, and networks have not been described in detail so as not to unnecessarily obscure aspects of the embodiments.

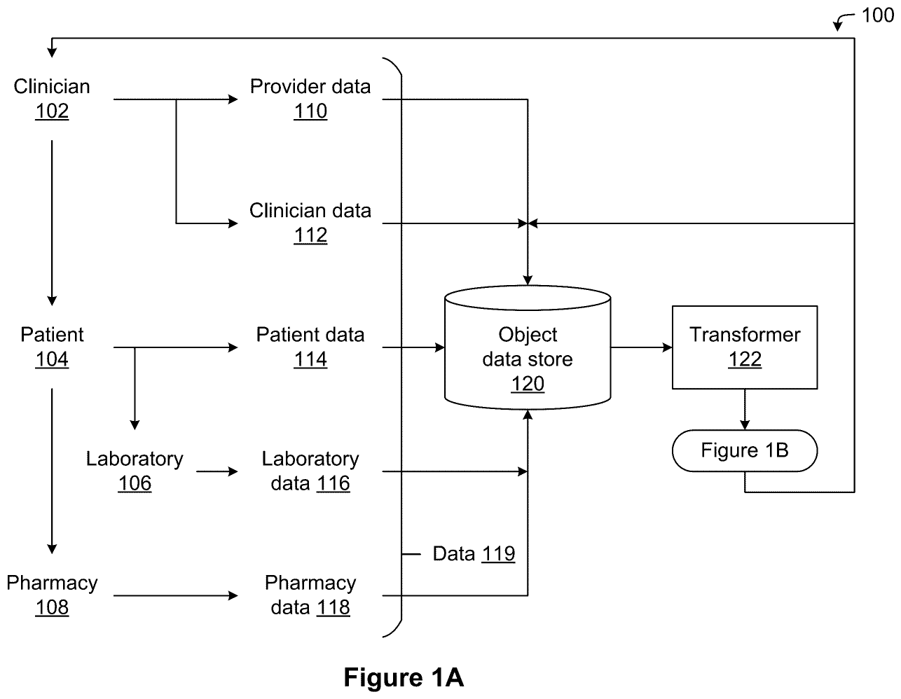

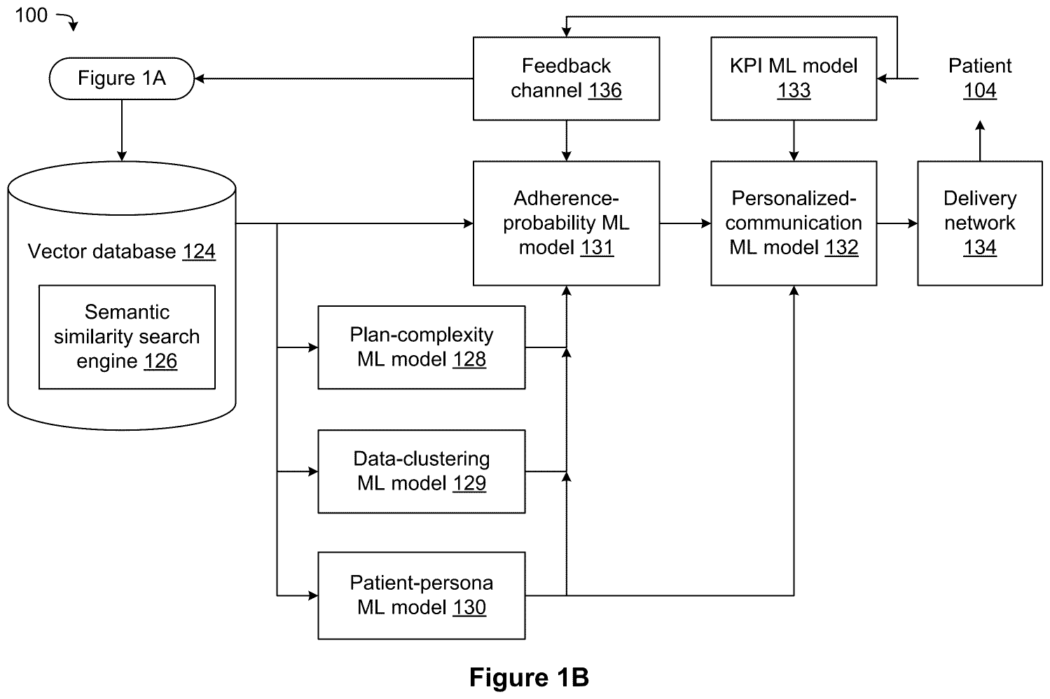

FIGS. 1A and 1B illustrate an exemplary system 100 for generating personalized communications regarding patient treatment plans, according to various aspects of the subject technology. At a high level, the example system 100 illustrated in FIGS. 1A and 1B: (i) receives data 119 regarding or relevant to a patient 104, (ii) uses a plurality of ML models 128-133 to construct a personalized communication based on the received data 119, where the personalized communication regards a treatment plan prescribed for the patient 104, and (iii) delivers the personalized communication to the patient 104 via a delivery network 134. As illustrated in FIGS. 1A and 1B, in some embodiments, the plurality of ML models includes many different ML models, each of which is discussed in more detail below. Given the complexity and nuanced level of detail needed to effectively generate a meaningful, personalized communication, a plurality of specialized ML models can prove more effective and/or capable than a single ML model configured to generate communications for a patient.

Examples of data types that can be used to train the plurality of ML models 128-133 and/or fed to the plurality of ML models 128-133 for generation of a personalized communication include data regarding: network tiering or network nuance leveraged, benefit complexity and customization, cost share complexity, complexity and magnitude of supplemental benefits included within the treatment plan (e.g., for plan-complexity ML model 128), communication preferences (e.g., language preference), aptitude for follow up via various stakeholders and/or channels, health-based on conditions and/or diagnoses given, and/or historical utilization of various types of benefits (e.g., for patient-persona ML model 130).

The plurality of ML models 128-133 may include, for instance, generative pre-trained transformer (GPT) models, convolutional neural networks (CNNs), support vector machines (SVMs), random forest models, and/or linear regression models. These ML models 128-133 can be trained prior to their use in the system 100 using data relevant to the respective tasks that the ML models 128-133 are configured to perform. These tasks and the ML models 128-133 are discussed in more detail below.

In the illustrated embodiment, data 119 from various sources are provided to an object data store 120, including provider data 110 and clinician data 112 from a clinician, patient data 114 from the patient 104, laboratory data 116 from a laboratory 106, and pharmacy data 118 from a pharmacy 108. The object data store 120 acts as a repository for the data 119.

The clinician 102, the laboratory 106, and/or the pharmacy 108 may be directly associated with the patient 104. For example, the clinician 102 may be a doctor, a dietician, or a surgeon that the patient 104 has visited in the past or has been instructed to visit in the future (e.g., as part of a treatment plan). Likewise, the laboratory 106 may be a laboratory at which the patient 104 has previously performed a medical exam or at which the patient 104 has been instructed to perform a medical exam (e.g., as part of a treatment plan). As a further example, the pharmacy 108 may be a pharmacy at which the patient 104 has filled a prescription or has been instructed to fill a prescription (e.g., as part of a treatment plan).

The provider data 110 and the clinician data 112 can be received or collected from the clinician 102. In some embodiments, the provider data 110 and/or the clinician data 112 regard an electronic medical record (EMR) of the patient 104, an electronic health record (EHR) of the patient 104, a treatment plan for the patient 104, and/or a diagnosis of the patient 104. The patient data 114 can be received or collected from the patient 104. In some embodiments, the patient data 114 regards demographics of the patient 104, an address of the patient 104, and/or a social media profile of the patient 104. The laboratory data 116 can be received or collected from a laboratory 106 that processes medical tests for the patient 104. In some embodiments, the laboratory data 116 regards a medical test order and/or a medical test result. The pharmacy data 118 can be received or collected from a pharmacy 108. In some embodiments, the pharmacy data 118 regards an average prescription processing time associated with the pharmacy 108 and/or a location of the pharmacy 108.

After receiving the data 119, in some embodiments, the object data store 120 stores the data 119 as objects. For example, the object data store 120 may leverage a JavaScript object notation (JSON)-like data model, with each data entity (e.g., an instance of provider data 110, clinician data 112, patient data 114, laboratory data 116, or pharmacy data 118) encapsulated in different serialized objects. Those objects can then be stored, for example, in an unordered storage pool and distributed across a commodity infrastructure. In some embodiments, each object is also assigned a unique identifier, further enabling the efficient retrieval and processing thereof.

Given that the clinician 102, the patient 104, the laboratory 106, and the pharmacy 108 may continue to produce data regarding or at least relevant to the patient 104, in some embodiments, the object data store 120 is scalable (e.g., horizontally scalable) and capable of growing to accommodate new data. Likewise, in some embodiments, the object data store 120 can shrink as old, irrelevant data is removed from the object data store 120.

In some embodiments, the object data store 120 may also use built-in replication, partitioning, or data protection mechanisms to increase reliability and availability. In some embodiments, a flexible schema-on-read architecture enables real-time analytics and AI workflows to generate actionable insights from multi-modal patient data. Further, in some embodiments, the object data store 120 flexibly manages unstructured or semi-structured data without requiring upfront schema definitions.

Additionally, in some embodiments, metadata tags are applied to the received data 119 in order to classify it. For example, metadata tags may indicate which of the data 119 is classified as provider data, clinician, patient data, and so on. Metadata may also include relevant identifiers, such as an identifier of the patient 104 or an identifier of an encounter with the patient 104. This can allow for more efficient filtering and analysis of the objects, which is discussed in more detail below. Moreover, metadata tags with identifiers indicative of a particular office visit or medical test can be used to associate objects stemming from the same office visit or medical test.

The data 119 stored in the object data store 120 (e.g., as objects) may be interconnected due to various relationships between the data 119. For example, objects that correspond to data of a same type (e.g., patient data 114) may be interconnected. As another example, objects corresponding to data regarding a particular period of time or a particular visit of the patient 104 to the clinician 102 may be interconnected. In some embodiments, relationships between the data 119 are managed using references rather than rigid schemas. Additionally, in some embodiments, new data types are added fluidly as needed to represent new data sources.

Downstream from the object data store 120 is the transformer 122. The transformer 122 is configured to transform data 119 (e.g., objects) from the object data store 120 into vector embeddings—numerical representations of the data 119 that capture semantic meaning embedded within it. For example, the vector embeddings may be high-dimensional vector representations capable of capturing nuanced relationships or similarities amongst the data 119, or capable of capturing connections between medical concepts represented in the data 119.

One of the benefits of transforming the data 119 into vector embeddings is that the transformation enables large-scale mining of the resultant vector embeddings. This mining process can uncover valuable patterns within high-dimensional data representations, generated through techniques like ML or natural language processing (NLP). For example, mining the vector embeddings may yield insights into the data 119 represented by the vector embeddings, such as insights into clinical notes, laboratory reports, and/or unstructured healthcare data. This is especially true for large data sets that regard, for example, multiple clinicians (e.g., including clinician 102), multiple patients (e.g., including patient 104), multiple laboratories (e.g., including laboratory 106), and/or multiple pharmacies (e.g., including pharmacy 108).

The transformer 122 may also be configured to filter the data 119 received from the object data store 120. This can occur before or after the transformation process. For example, the transformer 122 may remove identifiers from the data 119, translate abbreviations, normalize the data 119, and/or cluster together related portions of the data 119. Additionally, the transformer 122 can be configured to provide vector embeddings to an NLP model (e.g., a transformer-based NLP model), such as BERT or PubMedGPT. Afterwards, the transformer can then analyze contextual relationships between words or entities within the text of the data 119, for example, through a multi-headed self-attenuation mechanism.

Following transformation, the vector embeddings are provided from the transformer 122 to a vector database 124 (see FIG. 1B). In some embodiments, the vector embeddings are immutably appended to the vector database 124 according to the order in which they are generated. Metadata associated with the vector embeddings (e.g., document identifiers, patient identifiers, timestamps) may then be used to filter through the vector embeddings and/or join vector embeddings based on said metadata.

In some embodiments, the vector database 124 is a specialized, time-series vector database for efficient storage and retrieval of the vector embeddings. In contrast to traditional relational databases, storing the embeddings in a time-oriented vector database may significantly improve efficiency. This can accelerate both operational healthcare applications as well as analytical workloads for population health insights.

Moreover, in addition to the vector embeddings that correspond to the aforenoted data 119, the vector database 124 may also store vector embeddings corresponding to data regarding other clinicians, other patients, other laboratories, and/or other pharmacies. Storing vector embeddings together that regard multiple clinicians, patients, laboratories, and/or pharmacies may allow the system 100 to generate better personalized communications for the patient 104. For example, if it is determined that the data 119 regarding the patient is insufficient for generating a personalized communication or is otherwise lacking, the system 100 may retrieve additional data from the vector database to supplement the data 119.

In embodiments where the vector database 124 includes vector embeddings regarding multiple clinicians, multiple patients, multiple laboratories, and/or multiple pharmacies, a semantic search engine 126 can be used to parse through the vector database 124 to locate supplemental vector embeddings. Accordingly, the illustrated system 100 includes a semantic similarity search engine 126. For example, the system 100 can provide the vector embeddings corresponding to received data 119 to the semantic similarity search engine 126 as one or more query vectors, for searching the vector database 124. In doing so, the system 100 may locate vector embeddings in the vector database 124 that correspond to data similar to the received data 119 regarding the patient 104. Together with the received data 119, in some embodiments, the located data is also used in creating a personalized communication for the patient 104.

Vector embeddings located by the semantic similarity search engine 126 may correspond to clinicians, patients, laboratories, pharmacies, and/or medical contexts similar to those that generated the received data 119. These vector embeddings can provide a holistic view of potential risks, outcomes, and/or optimal interventions tailored to the patient's 104 nuanced profile. These vector embeddings can also forecast expected complexity, allowing for streamlined care coordination. The rich semantic insights derived from vector-similarity search thus enable fully personalized, evidence-based medicine, powering the next generation of healthcare artificial intelligence.

In identifying similar vector embeddings, the semantic similarity search engine 126 may compare the orientation of a query vector embedding against the orientations of other vector embeddings in the vector database 124. For example, in some embodiments, the semantic similarity search engine 126 employs a cosine-similarity search algorithm. Connections identified thereby may missed by rules-based search systems. Analyzing the neighborhood and aggregating details from these related patients provides invaluable context.

Following the vector database 124 and the semantic similarity search engine 126, the system 100 includes a plurality of ML models 128-133. In the illustrated embodiment, the system 100 includes a plan-complexity ML model 128, a data-clustering ML model 129, a patient-persona ML model 130, an adherence-probability ML model 131, a personalized-communication ML model 132, and a key-performance-indicator (KPI) ML model 133. In some embodiments, the system 100 does not include all of these ML models 128-133. Further, in some embodiments, the system 100 includes ML models in addition to the depicted ML models 128-133.

These ML models 128-133 are key in generating personalized communications for the patient 100. After the system 100 receives the aforenoted data 119 regarding the patient 104 (e.g., provider data 110, clinician data 112, patient data 114, laboratory data 116, and/or pharmacy data 118) and vectorizes the data 119 using the transformer 122, the system 100 provides the data 119 to the plurality of ML models 128-133 for generation of a personalized communication for the patient 104.

In some embodiments, the system 100 provides only the received data 119 to the ML models 128-133. For example, if the system 100 does not include a semantic similarity search engine, if the vector database does not include additional data, and/or if the received data 119 is sufficient for generating the personalized communication, then the system 100 may provide only the received data 119 to the ML models 128-133. However, in some embodiments, the system 100 provides the data 119 and additional data, retrieved using the semantic similarity search engine 126 (e.g., also as vector embeddings), to the plurality of ML models 128-133. In either instance, the ML models 128-133 are configured to (e.g., trained to) generate a personalized communication for the patient 104 based on data received from the vector database 124 (or, in some embodiments, directly from the object data store 120).

The ML models 128-133 work together to generate the personalized communication for the patient 104. Some of these ML models receive data directly from the vector database 124 (and/or the object data store 120), whereas other ML models receive output from upstream ML models. For example, in the illustrated embodiment, the adherence probability ML model 130 receives data directly from the vector database 124. By contrast, also in the illustrated embodiment, the personalized-communication ML model 132 receives the output of the adherence-probability ML model 131. These ML models 128-133 are discussed individually and in more detail hereinbelow.

Starting with the plan-complexity ML model 128, this ML model is configured to (e.g., trained to) determine a complexity level of a treatment plan prescribed for the patient 104. Higher treatment plan complexity is associated with lower treatment adherence due to an increased burden on the patient and a difficulty in coordinating care tasks. Patient communication and education is key to overcoming treatment plan complexity. Personalized communications, created to account for treatment plan complexity, can play an important role in communicating and educating the patient 104.

The complexity of a treatment plan may depend, for instance, on the number of medications prescribed in the treatment plan, the availability of said medications at local pharmacies, a dosing frequency associated with prescribed medications, side-effect management, administration means (e.g., oral, topical, intravenous), medication storage, titration or tapering of prescribed medications (e.g., starting the treatment with a particular dose and increasing or decreasing the dose with time), the number of chronic conditions being managed by the treatment plan, dietary restrictions, the presence of high-risk medications, necessary medical equipment, other special instructions, and so on. Data regarding any of these particular aspects of the treatment plan can be provided as inputs to the plan-complexity ML model 128.

Additionally, the plan-complexity ML model 128 can also receive data regarding similar treatment plans retrieved using the semantic similarity search engine 126. This data may be further informed by feedback received from patients associated with the similar treatment plans, where the feedback indicates whether those patients felt that their own treatment plans were complex and to what degree. This feedback can be extracted from surveys and quantified, for instance, by an ML model. Similarly, an ML model can be used to extract the aforenoted data regarding treatment plan complexity from the treatment plan prescribed for the patient 104.

In response to receiving the aforenoted data indicative of treatment plan complexity, in some embodiments, the plan-complexity ML model 128 produces a plan-complexity score that indicates the degree of complexity of the treatment plan prescribed for the patient 104. For example, the plan-complexity determination ML model 128 may return a “low complexity” score for a treatment plan involving less than three medications, with daily dosing requirements, and/or no high-risk medications. As another example, the plan-complexity ML model 128 may return a “medium complexity” score for a treatment plan involving three to five medications, and/or twice-per-day dosing requirements. As yet another example, the plan-complexity ML model 128 may return a “high complexity” score for a treatment plan involving more than five medications, frequent dosing requirements, multiple chronic conditions, and/or a high-risk medication.

The data-clustering ML model 129 also receives data directly from the vector database 124. This ML model serves to cluster the data into groups for risk analysis. For example, the data-clustering ML model 129 can cluster patient data (e.g., patient data 114 of FIG. 1A) into one group, provider data (e.g., provider data 110 of FIG. 1A) into another group, clinician data (e.g., clinician data 112 of FIG. 1A) into yet another group, and so on for laboratory data (e.g., laboratory data 116 of FIG. 1A) and pharmacy data (e.g., pharmacy data 118 of FIG. 1A). It is noted that in embodiments where only the received data 119 is provided to the ML models 128-133, the system 100 may not include a data-clustering ML model 129.

From there, the data-clustering ML model 129 may further group the data into sub-clusters, for instance, based on various aspects of the data that may impact treatment adherence probabilities. This is discussed in more detail below with respect to each particular type of data. At a high level, the objective in clustering the data is to identify which combinations of patient groups, provider groups, pharmacy groups, and so on might pose an adherence risk for a particular treatment plan (e.g., based on the complexity of the treatment plan). Clustering is done to return groups of similar patient, provider, pharmacy, and/or laboratory data.

For patient data, the data can be grouped based on factors including age, mental health, health literacy, disabilities, socio-economic status, and/or cultural beliefs. These factors may impact the ability of a patient to adhere to and manage a complex treatment plan. High-patient risk may mean that the treatment plan should be simplified or that the personalized communication should be more robust.

For provider data, the data can be grouped based on factors including provider communication skills, patient satisfaction, time constraints, care fragmentation, deficient knowledge, provider outcomes, and/or provider dismissiveness. These factors may impact treatment plan design. For example, less-skilled providers may create treatment plans that do not match patient capabilities or integrate with the patient's lifestyle. High provider risk is associated with overly complex treatment plans.

For pharmacy data, the data can be grouped based on factors including operating hours, drug costs, drugs sold, pharmacy rating and reviews, pharmacy organization, delivery options and feeds, accepted insurance plans, pharmacy type (e.g., independent, chain, clinic, mail order), medicine stick level, generic drug options, automated refill options, whether the pharmacy has inventory issues, and/or whether the pharmacy offer automatic reminders for the patient (e.g., text-message based reminders). These factors may indicate a greater chance of disruption for the patient in following a prescribed treatment plan that involves one or more medications.

For laboratory data, the data can be grouped based on factors including location, operating hours, demand, communication gaps, laboratory ratings or reviews, appointment flexibility, wait times, specialty tests offered, costs of tests, accepted insurance plans, test methodology, online patient portal access, notification option for result availability, integration with EMR systems, and time to receive test results. These factors may prevent the patient from completing laboratory work to inform the treatment plan. High risk for the laboratory may mean less reliability for laboratory-dependent treatment plans.

The next ML model, the patient-persona ML model 130, is configured to (e.g., trained to) generate persona indicators for the patient that indicate various aspects of the patient's persona. For example, the persona indicators may indicate an organization level of the patient, an ability of the patient to follow a prescribed regime, a communication style of the patient, how well the patient responds to tracking tools such as calendars, whether the patient prefers factual information in discussing medical matters, whether the patient tends to be skeptical, whether the patient prefers two-way, open-ended communication, whether the patient responds well to empathy from healthcare providers, whether the patient tends to be rational, whether the patient is worried about side effects of the treatment plan, and/or a degree to which the patient is open to lifestyle adjustments. The ultimate goal of the patient-personal ML 130 model is to determine what motivates the patient 104 so that the personalized communication can be generated in a manner that the patient will respond positively to.

In generating these persona indicators, the patient-persona ML model 130 may require data regarding an EMR of the patient, an EHR of the patient, social determinants of health (SDOH) for the patient, and/or a social media profile of the patient. In some embodiments, the vectorized embedding data (e.g., EMR, EHR, SDOH, and/or social media data) from the vector database 124 can be clustered using an unsupervised learning algorithm. These clusters can then be analyzed to discover facts like communication preferences, adherence patterns, and other persona indicators.

The plan-complexity and patient-persona ML models 128 and 130 (and, in some embodiments, the data-clustering ML model 129) provide their outputs to the adherence-probability ML model 131. The adherence-probability ML model 131 is configured to (e.g., trained to) generate an adherence-probability score that indicates a probability of the patient 104 adhering to the treatment plan. This score may be based on the plan-complexity score, the persona indicators, data clusters, and/or additional data from the vector database, such as data regarding the particulars of the treatment plan. The adherence-probability score can be used in generating the personalized communication. For example, if the probability of adherence is high, then the personalized communication may not need to be as in-depth, educational, or tailored to the patient as it would otherwise need to be. In some embodiments, a supervised learning algorithm (e.g., RandomForest) is used for training on a similar dataset to determine the likelihood of medication adherence based on different situations.

In some embodiments, the adherence-probability ML model 131 is also configured to (e.g., trained to) generate a communication plan for the personalized communication. The communication plan may indicate, for example, whether the personalized communication should include text, images, or video and/or whether the personalized communication should be delivered via email, text message, notification, phone call, or letter. As with the adherence-probability score, the adherence-probability ML model can generate the communication plan based on the plan-complexity score, data clusters, persona indicators, and/or data from the vector database 124 (e.g., regarding the treatment plan). In some embodiments, the personalized-communication ML model 132 is configured to generate the communication plan rather than the adherence-probability ML model 131. Alternatively, in some implementations, the personalized-communication ML model 132 is configured to supplement the communication plan after receiving it from the adherence-probability ML model 131.

Regardless of where the communication plan is generated, it can play an important role in improving patient treatment plan adherence. The goal with the communication plan is to present the personalized communication in a manner that the patient 104 will best respond to, while also accommodating patient preferences and the need to educate the patient and account for the complexity of the treatment plan. A flexible, personalized communication plan with active monitoring of patient adherence is key to improving adherence to the treatment plan. In this manner, the plan-complexity, data-clustering, and/or patient-persona ML models 128-130 can act as helper ML models, selectively providing data (e.g., regarding the patient and/or similar patients) and scores to the adherence-probability ML model 131 (or, in some embodiments, the personalized-communication ML model 132).

The adherence-probability ML model 131 may also receive data from a feedback channel 136, as illustrated in the example system 100. This functionality, as well as the feedback channel 136, is discussed in more detail below.

Turning now to the main ML model, the personalized-communication ML model 132 is configured to (e.g., trained to) generate the personalized communication for the patient. In some embodiments, the personalized communication is generated based on the adherence-probability score and the persona indicators. Generating the personalized communication may also be based on the communication plan, regardless of whether the communication plan is generated by the adherence-probability ML model 131 or the personalized-communication ML model 132. Further, generating the personalized communication may be based on data from the other ML models (e.g., the plan-complexity score) and/or the vector database 124, such as data regarding the treatment plan or data regarding the patient 104, the clinician 102, the laboratory 106, and/or the pharmacy 108. Moreover, generating the personalized communication may be based on example communications from the clinician 102 and/or other clinicians regarding the treatment plan for the patient and/or other treatment plans.

In some embodiments, the personalized-communication ML model 132 is a GPT-based model to leverage GPT's natural language proficiency and adaptability to dynamically create adherence content optimized for the patient's 104 needs and/or communication preferences. For example, the personalized-communication ML model 132 may vary the reading level, sentence complexity, tone, vocabulary, language, terminology needs, and/or audio or video or animation of the personalized communication. In some embodiments, the personalized-communication ML model 132 also includes a large language model (LLM) and/or a stable-diffusion model for synthesizing content creatively personalized to the needs of the patient 104 and/or to the treatment plan.

The personalized communication may be addressed directly to the patient 104. Additionally, the personalized communication may refer to specific aspects of the patient treatment plan. Further, the personalized communication may include one or more of video, audio, text, and/or animation (e.g., as specified by the communication plan), which can then be fed to the delivery network 132 for delivery to the patient 104.

The delivery network 134 is configured to provide the personalized communication to the patient, and it can include a variety of communication channels. For example, the delivery network may include digital channels, such as a payor portal, a provider portal, social media, text messaging, email, notifications (e.g., at a smartphone, a smartwatch, and/or a fitness tracker), personalized advertising, and/or an EMR. As another example, the delivery network may include physical channels, such as face-to-face communication (e.g., between the clinician 102 and the patient 104), phone calls, care-management programs, physical mail, and/or health hardware. In some embodiments, the delivery network is further configured to provide the personalized communication to the clinician 102, the laboratory 106, the pharmacy 108, and/or another third-party for relaying to the patient 104 (e.g., at a next doctor's visit).

In some embodiments, the aforenoted communication plan determines which channel of the delivery network 134 should be used to deliver the personalized communication to the patient (or to a third party for relay to the patient). In this manner, the delivery network 134 can be leveraged to deliver the personalized communication to the patient (or to a third party) to maximize the patient's 104 reception of the personalized communication.

After the delivery network 134 delivers the personalized communication, the KPI ML model 133 is used to track patient 104 adherence and determine the impact of the personalized communication on the patient 104. In some embodiments, a baseline adherence metric is created in order to measure the impact of the personalized communication. For example, the baseline adherence metric may account for missed doses, possession ratio, discontinuation rate, and other such factors. The KPI ML model 133 can thus measure clinical markers affected by adherence and determine how said markers are affected by the personalized communication.

The KPI ML model 133 can be configured to (e.g., trained to) generate recommendations for the personalized-communication ML model 132 based on how the patient responds to the personalized communication. The personalized-communication ML model 132 can then use these recommendations to refine future personalized communications for the patient 104.

Furthermore, a feedback channel 136 can also be used to refine future personalized communications, retrain the ML models 128-133, and/or inform the clinician 102, the laboratory 106, and/or the pharmacy 108. In some embodiments, the feedback channel 136 is configured to receive feedback (e.g., direct feedback and/or indirect feedback) from the patient regarding the personalized communication. For example, the feedback may indicate an amount of the personalized communication reviewed by the patient, a response of the patient to the personalized communication, and/or an affect of the personalized communication on adherence to the treatment plan by the patient.

After receiving this feedback, the feedback channel 136 can then provide the feedback to the clinician 102 and/or the adherence probability ML model 131 (or, in some embodiments, to the personalized-communication ML model 132). Accordingly, the adherence-probability ML model 131 (or the personalized-communication ML model 132) can be configured to receive the feedback and generate future adherence-probability scores (or communication plans and/or personalized communications) based on the feedback from the feedback channel 136.

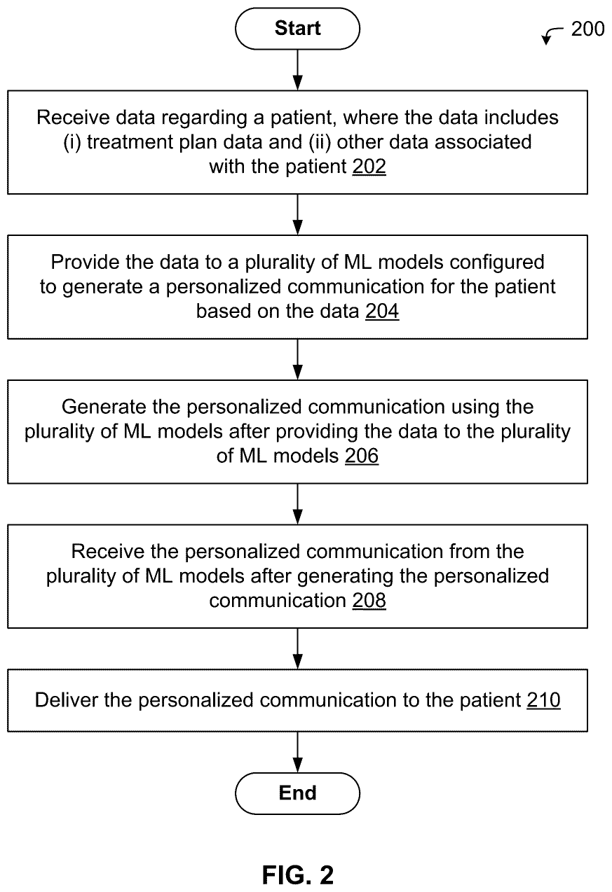

FIG. 2 illustrates an example process 200 for improving adherence to a patient treatment plan, according to various aspects of the subject technology. The process 200 can be implemented, for example, by one or more computing devices, such as one or more processors of one or more electronic devices. An example electronic device for implementation of the example process 200 may include a non-transitory, computer-readable storage medium (e.g., random access memory, RAM) storing instructions that, when executed by a processor of the electronic device, cause the electronic device to perform one or more operations of the process 200.

In some embodiments, one or more of the operations are implemented based on one or more ML algorithms. Moreover, in some embodiments, one or more of the operations are implemented apart from other operations and by one or more different processors or devices. Further, for explanatory purposes, the operations of the process 200 are described as occurring in serial (i.e., linearly). However, some of the operations of the process 200 can occur in parallel (i.e., simultaneously). Additionally, the operations of the process 200 need not be performed in the order shown and, in some embodiments, one or more of the operations need not be performed whatsoever. Moreover, it is noted that the present disclosure used the term “configured to” to describe the capabilities of various ML models. In some instances, this term means “trained to” when used in the context of ML models.

In the illustrated embodiment, the process 200 includes receiving (202) data regarding a patient (e.g., patient 104 of FIGS. 1A-1B). The data includes treatment plan data regarding a treatment plan prescribed for the patient, as well as other data regarding (i) a healthcare provider associated with the patient (e.g., provider data 112 of FIGS. 1A-1B), (ii) a clinician (e.g., clinician 102 of FIGS. 1A-1B) associated with the patient (e.g., clinician data 112 of FIGS. 1A-1B), (iii) a medical record (e.g., an EMR, an EHR) associated with the patient (e.g., patient data 114 of FIGS. 1A-1B), (iv) a social media account associated with the patient (e.g., patient data 114 of FIGS. 1A-1B), (v) a laboratory (e.g., laboratory 106 of FIGS. 1A-1B) associated with the patient (e.g., laboratory data 116 of FIGS. 1A-1B), and/or (vi) a pharmacy (e.g., pharmacy 108 of FIGS. 1A-1B) associated with the patient (e.g., pharmacy data 118 of FIGS. 1A-1B).

The process 200 also includes providing (204) the data to one or more ML models of a plurality of ML models (e.g., ML models 128-133 of FIGS. 1A-1B), where the plurality of ML models is configured to generate a personalized communication for the patient based on the data. Additionally, the process 200 includes generating (206) the personalized communication using the plurality of ML models after providing the data to the plurality of ML models. Further, the process 200 includes receiving (208) the personalized communication from the plurality of ML models after generating the personalized communication. The personalized communication may be addressed to the patient and/or regard the treatment plan prescribed for the patient. Moreover, the process 200 includes delivering (210) the personalized communication to the patient (e.g., via delivery network 134 of FIGS. 1A-1B).

As illustrated above, various types of data (e.g., data 119 of FIGS. 1A-1B) can be used in generating the personalized communication. This data may need to be stored prior to providing it to the plurality of ML models. Thus, in some embodiments, the process 200 also includes storing the data regarding the patient in a storage layer (e.g., including object data store 120 and/or vector database 124 of FIGS. 1A-1B). Additionally, the process 200 may include receiving additional data regarding additional patients and storing the additional data regarding the additional patients in the storage layer. Further, the process 200 may include determining that the data should be supplemented with the additional data. Moreover, the process 200 may include, responsive to determining that the data should be supplemented, (i) retrieving a relevant portion of the additional data using a search engine (e.g., semantic search engine 126 of FIGS. 1A-1B) and (ii) providing the relevant portion of the additional data to the plurality of ML models prior to receiving the personalized communication. The search engine may be configured to receive a portion of the data as a query and locate the relevant portion of the additional data that is similar to the query. Furthermore, the plurality of ML models generating the personalized communication may further be based on the relevant portion of the additional data.

The plurality of ML models may include a data-clustering ML model (e.g., data-clustering ML model 129 of FIGS. 1A-1B). In some embodiments, the data-clustering ML model is configured to receive the data and the relevant portion of the additional data (e.g., retrieve by the search engine) and generate data clusters of the data and the relevant portion of the additional data. The data clusters may be grouped according to data type and/or risk of non-adherence. Accordingly, providing the data to the plurality of ML models may include providing the data to the data-clustering ML model, and providing the relevant portion of the additional data to the plurality of ML models may include providing the relevant portion of the additional data to the data-clustering ML model. Further, the plurality of ML models generating the personalized communication may further be based on the data clusters.

The plurality of ML models may also include a plan-complexity ML model (e.g., plan-complexity ML model 128 of FIGS. 1A-1B). In some embodiments, the plan-complexity ML model is configured to receive the treatment plan data and generate a plan-complexity score based on the treatment plan data. The treatment plan data can include, for example, (i) a number of medications prescribed in the treatment plan, (ii) an availability of the prescribed medications, (iii) a dosage frequency for the prescribed medications, (iv) a number of chronic conditions addressed by the treatment plan, (v) an indication that one or more of the prescribed medications is a high-risk medication, (vi) a dietary restriction prescribed in the treatment plan, and (vii) an indication that the prescribed treatment plan requires special medical equipment. Moreover, the plan-complexity score can indicate a complexity level of the treatment plan prescribed for the patient. Accordingly, providing the data to the plurality of ML models may include providing the treatment plan data to the plan-complexity ML model, and the plurality of ML models generating the personalized communication may further be based on the treatment-plan-complexity score.

Additionally, the plurality of ML models may include a patient-persona ML model (e.g., patient-persona ML model 130 of FIGS. 1A-1B). In some embodiments, the patient-persona ML model is configured to receive the other data and generate persona indicators based on the other data. The other data may at least include, for example, the medical record associated with the patient and/or the social media account associated with the patient (e.g., social media posts). Moreover, the persona indicators may indicate, for example, an organization level of the patient, an ability of the patient to follow a prescribed regime, and/or a communication style of the patient. Accordingly, providing the data to the plurality of ML models may include providing the other data to the patient-persona ML model, and the plurality of ML models generating the personalized communication may further be based on the persona indicators.

Moreover, the plurality of ML models may include an adherence-probability ML model (e.g., adherence-probability ML model 131 of FIGS. 1A-1B). In some embodiments, the adherence-probability ML model is configured to receive the treatment plan data, receive the plan-complexity score from the plan-complexity ML model, receive the persona indicators from the patient-persona ML model, and generate an adherence-probability score for the patient based on the treatment plan data, the plan-complexity score, and the persona indicators. The adherence-probability score may indicate, for example, a probability of the patient adhering to the treatment plan. Accordingly, providing the data to the plurality of ML models comprises providing the treatment plan data to the adherence-probability ML model, the plan-complexity ML model may be further configured to provide the plan-complexity score to the adherence-probability ML model, and the patient-persona ML model may be further configured to provide the persona indicators to the adherence probability ML model. Moreover, the plurality of ML models generating the personalized communication may further be based on the adherence-probability score.

Furthermore, the plurality of ML models may include a personalized-communication ML model (e.g., personalized-communication ML model 132 of FIGS. 1A-1B). In some embodiments, the personalized-communication ML model is configured to receive the treatment plan data, receive the adherence-probability score from the adherence probability ML model, receive the persona indicators from the patient-persona ML model, and generate the personalized communication based on the treatment plan data, the adherence-probability score, and the persona indicators. Accordingly, providing the data to the plurality of ML models may include providing the treatment plan data to the personalized-communication ML model. Additionally, the adherence-probability ML model may further be configured to provide the adherence-probability score to the personalized-communication ML model, and the patient-persona ML model may further be configured to provide the persona indicators to the personalized-communication ML model.

A feedback channel can be used to improve future personalized communications generated using the process 200. Accordingly, in some embodiments, the process 200 includes receiving feedback from the patient regarding the personalized communication. The feedback may indicate, for example, an amount of the personalized communication reviewed by the patient, a response of the patient to the personalized communication, and/or an affect of the personalized communication on adherence to the treatment plan by the patient. The process 200 may also include providing the feedback to the clinician and the adherence-probability ML model (and/or the personalized-communication ML model), wherein the adherence-probability ML model is further configured to receive the feedback, and wherein the adherence-probability ML model generating the adherence-probability score is further based on the feedback. Additionally, the process 200 may include receiving a new personalized communication from the plurality of ML models responsive to providing the data to the plurality of ML models and responsive to providing the feedback to the adherence-probability ML model.

The ML models can further be used to determine a communication plan for deliver of the personalized communication. For example, the personalized communication may indicate whether the personalized communication should include text, images, or video and/or whether the personalized communication should be delivered via email, text message, notification, phone call, or letter. Accordingly, in some embodiments, the personalized-communication ML model (and/or the adherence-probability ML model) is configured to generate a communication plan based on the treatment plan data, the adherence-probability score, and/or the persona indicators. The personalized ML model generating the personalized communication may further be based on the communication plan. Additionally, delivering the personalized communication to the patient may include delivering the personalized communication according to the communication plan.

### Illustration of Subject Technology as Clauses

Various examples of aspects of the present disclosure are described as numbered clauses below. These are provided as examples and are not intended limit the subject technology. Identifications of the figures and reference numbers are provided below merely as examples and for illustrative purposes, and the clauses are not limited by these identifications.

Clause 1. A system for improving adherence to patient treatment plans, the system comprising: a transformer configured to vectorize data regarding a patient; a storage layer configured to store the data; a plurality of machine learning (ML) models configured to generate a personalized communication for the patient based on the data, wherein the plurality of ML models comprises (i) a plan-complexity ML model trained using historical adherence data associated with treatment plans similar to a treatment plan prescribed for the patient, (ii) a patient-persona ML model trained using behavioral pattern data associated with patients similar to the patient, (iii) an adherence-probability ML model trained using the historical adherence data and persona data associated with patients similar to the patient, and (iv) a personalized-communication ML model trained using interaction data comprising language from interactions involving other patients similar to the patient; a delivery network configured to deliver the personalized communication to the patient; and an electronic device comprising a processor configured to: receive the data regarding the patient, wherein the data comprises (i) treatment plan data regarding a treatment plan prescribed for the patient, and (ii) other data regarding a healthcare provider associated with the patient, a clinician associated with the patient, a medical record associated with the patient, a social media account associated with the patient, a laboratory associated with the patient, or a pharmacy associated with the patient; vectorize the data using the transformer, wherein vectorizing the data comprises translating the treatment plan data and the other data into numerical representations that capture semantic meaning embedded within the treatment plan data and the other data; store the data in the storage layer after vectorizing the data; provide the treatment plan data from the storage layer to the plan-complexity ML model, wherein the treatment plan data comprises (i) a number of medications prescribed in the treatment plan, and (ii) a dosage frequency for the prescribed medications; receive a plan-complexity score from the plan-complexity ML model responsive to providing the treatment plan data to the plan-complexity ML model, wherein the plan-complexity score indicates a complexity level of the treatment plan; provide the other data from the storage layer to the patient-persona ML model, wherein the other data regards at least (i) the medical record associated with the patient and (ii) the social media account associated with the patient; receive persona indicators from the patient-persona ML model responsive to providing the other data to the patient-persona ML model, wherein the persona indicators indicate (i) an ability of the patient to follow a prescribed regime and (ii) a communication style of the patient; provide the treatment plan data from the storage layer, the plan-complexity score, and the persona indicators to the adherence-probability ML model; receive an adherence-probability score from the adherence-probability ML model responsive to providing the treatment plan data, the plan-complexity score, and the persona indicators to the adherence-probability ML model, wherein the adherence-probability score indicates a probability of the patient adhering to the treatment plan; provide the treatment plan data from the storage layer, the adherence-probability score, and the persona indicators to the personalized-communication ML model; receive a communication plan and the personalized communication from the personalized-communication ML model, wherein (i) the communication plan indicates whether the personalized communication should include text, images, or video and whether the personalized communication should be delivered via email, text message, notification, phone call, or letter and (ii) the personalized communication is addressed to the patient and regards the treatment plan prescribed for the patient; and deliver the personalized communication to the patient via the delivery network and according to the communication plan.

Clause 2. An electronic device configured to generate personalized communications regarding patient treatment plans, the electronic device comprising a processor configured to: receive data regarding a patient, wherein the data comprises (i) treatment plan data regarding a treatment plan prescribed for the patient and (ii) other data regarding a healthcare provider associated with the patient, a clinician associated with the patient, a medical record associated with the patient, a social media account associated with the patient, a laboratory associated with the patient, or a pharmacy associated with the patient; provide the data to a plurality of ML models configured to generate a personalized communication for the patient based on the data; receive the personalized communication from the plurality of ML models responsive to providing the data to the plurality of ML models, wherein the personalized communication is addressed to the patient and regards the treatment plan prescribed for the patient; and deliver the personalized communication to the patient.

Clause 3. The electronic device of Clause 2, wherein the plurality of ML models comprises a plan-complexity ML model configured to: receive the treatment plan data, wherein the treatment plan data comprises three or more of (i) a number of medications prescribed in the treatment plan, (ii) an availability of the prescribed medications, (iii) a dosage frequency for the prescribed medications, (iv) a number of chronic conditions addressed by the treatment plan, (v) an indication that one or more of the prescribed medications is a high-risk medication, (vi) a dietary restriction prescribed in the treatment plan, and (vii) an indication that the prescribed treatment plan requires special medical equipment; and generate a plan-complexity score based on the treatment plan data, wherein the plan-complexity score indicates a complexity level of the treatment plan; wherein (i) providing the data to the plurality of ML models comprises providing the treatment plan data to the plan-complexity ML model and (ii) the plurality of ML models generating the personalized communication is further based on the treatment-plan-complexity score.

Clause 4. The electronic device of either Clause 2 or Clause 3, wherein the plurality of ML models comprises a patient-persona ML model configured to: receive the other data, wherein the other data regards at least (i) the medical record associated with the patient and (ii) the social media account associated with the patient; and generate persona indicators based on the other data, wherein the persona indicators indicate two or more of (i) an organization level of the patient, (ii) an ability of the patient to follow a prescribed regime, and (iii) a communication style of the patient; wherein (i) providing the data to the plurality of ML models comprises providing the other data to the patient-persona ML model and (ii) the plurality of ML models generating the personalized communication is further based on the persona indicators.

Clause 5. The electronic device of Clauses 3 and 4, wherein the plurality of ML models further comprises an adherence-probability ML model configured to: receive the treatment plan data; receive the plan-complexity score from the plan-complexity ML model; receive the persona indicators from the patient-persona ML model; and generate an adherence-probability score for the patient based on the treatment plan data, the plan-complexity score, and the persona indicators, wherein the adherence-probability score indicates a probability of the patient adhering to the treatment plan; wherein (i) providing the data to the plurality of ML models comprises providing the treatment plan data to the adherence-probability ML model, (ii) the plan-complexity ML model is further configured to provide the plan-complexity score to the adherence-probability ML model, and (iii) the patient-persona ML model is further configured to provide the persona indicators to the adherence probability ML model, and (iv) the plurality of ML models generating the personalized communication is further based on the adherence-probability score.

Clause 6. The electronic device of Clause 5, further comprising a feedback channel configured to: receive feedback from the patient regarding the personalized communication, wherein the feedback indicates two or more of (i) an amount of the personalized communication reviewed by the patient, (ii) a response of the patient to the personalized communication, and (iii) an affect of the personalized communication on adherence to the treatment plan by the patient; and provide the feedback to (i) the clinician and (ii) the adherence-probability ML model, wherein the adherence-probability ML model is further configured to receive the feedback, and wherein the adherence-probability ML model generating the adherence-probability score is further based on the feedback.

Clause 7. The electronic device of either Clause 5 or Clause 6, wherein the plurality of ML models further comprises a personalized-communication ML model configured to: receive the treatment plan data; receive the adherence-probability score from the adherence-probability ML model; receive the persona indicators from the patient-persona ML model; and generate the personalized communication based on the treatment plan data, the adherence-probability score, and the persona indicators; wherein (i) providing the data to the plurality of ML models comprises providing the treatment plan data to the personalized-communication ML model, (ii) the adherence-probability ML model is further configured to provide the adherence-probability score to the personalized-communication ML model, and (iii) the patient-persona ML model is further configured to provide the persona indicators to the personalized-communication ML model.

Clause 8. The electronic device of Clause 7, wherein the personalized-communication ML model is further configured to: prior to generating the personalized communication, generate a communication plan based on the treatment plan data, the adherence-probability score, and the persona indicators, wherein the communication plan indicates (i) whether the personalized communication should include text, images, or video and (ii) whether the personalized communication should be delivered via email, text message, notification, phone call, or letter; wherein the personalized-communication ML model generating the personalized communication is further based on the communication plan, and delivering the personalized communication to the patient comprises delivering the personalized communication according to the communication plan.

Clause 9. The electronic device of Clause 2, further comprising: a storage layer configured to (i) store the data regarding the patient and (ii) store additional data regarding additional patients; and a search engine configured to (i) receive a portion of the data as a query and (ii) locate a relevant portion of the additional data that is similar to the query; wherein the electronic device is further configured to (i) store the data in the storage layer, (ii) receive the additional data, (iii) store the additional data in the storage layer, (iv) determine that the data should be supplemented with the additional data, and (v) responsive to determining that the data should be supplemented, retrieve the relevant portion of the additional data using the search engine and provide the relevant portion of the additional data to the plurality of ML models prior to receiving the personalized communication; and wherein the plurality of ML models generating the personalized communication is further based on the relevant portion of the additional data.

Clause 10. The electronic device of Clause 9, wherein the plurality of ML models comprises a data-clustering ML model configured to: receive the data and the relevant portion of the additional data; and generate data clusters of the data and the relevant portion of the additional data, wherein the data clusters are grouped according to data type and risk of non-adherence; wherein (i) providing the data to the plurality of ML models comprises providing the data to the data-clustering ML model, (ii) providing the relevant portion of the additional data to the plurality of ML models comprises providing the relevant portion of the additional data to the data-clustering ML model, and (ii) the plurality of ML models generating the personalized communication is further based on the data clusters.

Clause 11. The electronic device of either Clause 9 or Clause 10, wherein the storage layer comprises: an object data store configured to store the data regarding the patient and the additional data regarding the additional patients; a transformer configured to vectorize the data and the additional data; and a vector database configured to store the data and the additional data after the transformer vectorizes the data and the additional data; wherein the query comprises a vectorized portion of the data, and the search engine retrieving the relevant portion of the additional data comprises the search engine retrieving the relevant portion of the additional data from the vector database.

Clause 12. A computer-implemented method for improving adherence to patient treatment plans, the method comprising: receiving data regarding a patient, wherein the data comprises (i) treatment plan data regarding a treatment plan prescribed for the patient, and (ii) other data regarding a healthcare provider associated with the patient, a clinician associated with the patient, a medical record associated with the patient, a social media account associated with the patient, a laboratory associated with the patient, or a pharmacy associated with the patient; providing the data to a plurality of ML models configured to generate a personalized communication for the patient based on the data; generating the personalized communication using the plurality of ML models after providing the data to the plurality of ML models, wherein the personalized communication is addressed to the patient and regards the treatment plan prescribed for the patient; receiving the personalized communication from the plurality of ML models after generating the personalized communication; and delivering the personalized communication to the patient.

Clause 13. The computer-implemented method of Clause 12, wherein the plurality of ML models comprises a plan-complexity ML model configured to: receive the treatment plan data, wherein the treatment plan data comprises three or more of (i) a number of medications prescribed in the treatment plan, (ii) an availability of the prescribed medications, (iii) a dosage frequency for the prescribed medications, (iv) a number of chronic conditions addressed by the treatment plan, (v) an indication that one or more of the prescribed medications is a high-risk medication, (vi) a dietary restriction prescribed in the treatment plan, and (vii) an indication that the prescribed treatment plan requires special medical equipment; and generate a plan-complexity score based on the treatment plan data, wherein the plan-complexity score indicates a complexity level of the treatment plan; wherein (i) providing the data to the plurality of ML models comprises providing the treatment plan data to the plan-complexity ML model and (ii) the plurality of ML models generating the personalized communication is further based on the treatment-plan-complexity score.

Clause 14. The computer-implemented method of either Clause 12 or Clause 13, wherein the plurality of ML models comprises a patient-persona ML model configured to: receive the other data, wherein the other data regards at least (i) the medical record associated with the patient and (ii) the social media account associated with the patient; and generate persona indicators based on the other data, wherein the persona indicators indicate (i) an organization level of the patient, (ii) an ability of the patient to follow a prescribed regime, and (iii) a communication style of the patient; wherein (i) providing the data to the plurality of ML models comprises providing the other data to the patient-persona ML model and (ii) the plurality of ML models generating the personalized communication is further based on the persona indicators.

Clause 15. The computer-implemented method of Clause 13 and Clause 14, wherein the plurality of ML models further comprises an adherence-probability ML model configured to: receive the treatment plan data; receive the plan-complexity score from the plan-complexity ML model; receive the persona indicators from the patient-persona ML model; and generate an adherence-probability score for the patient based on the treatment plan data, the plan-complexity score, and the persona indicators, wherein the adherence-probability score indicates a probability of the patient adhering to the treatment plan; wherein (i) providing the data to the plurality of ML models comprises providing the treatment plan data to the adherence-probability ML model, (ii) the plan-complexity ML model is further configured to provide the plan-complexity score to the adherence-probability ML model, and (iii) the patient-persona ML model is further configured to provide the persona indicators to the adherence probability ML model, and (iv) the plurality of ML models generating the personalized communication is further based on the adherence-probability score.

Clause 16. The computer-implemented method of Clause 15, further comprising: receiving feedback from the patient regarding the personalized communication, wherein the feedback indicates two or more of (i) an amount of the personalized communication reviewed by the patient, (ii) a response of the patient to the personalized communication, and (iii) an affect of the personalized communication on adherence to the treatment plan by the patient; and providing the feedback to (i) the clinician and (ii) the adherence-probability ML model, wherein the adherence-probability ML model is further configured to receive the feedback, and wherein the adherence-probability ML model generating the adherence-probability score is further based on the feedback.

Clause 17. The computer-implemented method of either Clause 15 or Clause 16, wherein the plurality of ML models further comprises a personalized-communication ML model configured to: receive the treatment plan data; receive the adherence-probability score from the adherence-probability ML model; receive the persona indicators from the patient-persona ML model; and generate the personalized communication based on the treatment plan data, the adherence-probability score, and the persona indicators; wherein (i) providing the data to the plurality of ML models comprises providing the treatment plan data to the personalized-communication ML model, (ii) the adherence-probability ML model is further configured to provide the adherence-probability score to the personalized-communication ML model, and (iii) the patient-persona ML model is further configured to provide the persona indicators to the personalized-communication ML model.

Clause 18. The computer-implemented method of Clause 17, wherein the personalized-communication ML model is further configured to: prior to generating the personalized communication, generate a communication plan based on the treatment plan data, the adherence-probability score, and the persona indicators, wherein the communication plan indicates (i) whether the personalized communication should include text, images, or video and (ii) whether the personalized communication should be delivered via email, text message, notification, phone call, or letter; wherein the personalized-communication ML model generating the personalized communication is further based on the communication plan, and delivering the personalized communication to the patient comprises delivering the personalized communication according to the communication plan.

Clause 19. The computer-implemented method of Clause 12, further comprising: storing the data regarding the patient in a storage layer; receiving additional data regarding additional patients; storing the additional data regarding the additional patients in the storage layer; determining that the data should be supplemented with the additional data; responsive to determining that the data should be supplemented, (i) retrieving a relevant portion of the additional data using a search engine and (ii) providing the relevant portion of the additional data to the plurality of ML models prior to receiving the personalized communication; wherein the search engine is configured to receive a portion of the data as a query and locate the relevant portion of the additional data that is similar to the query, and the plurality of ML models generating the personalized communication is further based on the relevant portion of the additional data.

Clause 20. The electronic device of Clause 19, wherein the plurality of ML models comprises a data-clustering ML model configured to: receive the data and the relevant portion of the additional data; and generate data clusters of the data and the relevant portion of the additional data, wherein the data clusters are grouped according to data type and risk of non-adherence; wherein (i) providing the data to the plurality of ML models comprises providing the data to the data-clustering ML model, (ii) providing the relevant portion of the additional data to the plurality of ML models comprises providing the relevant portion of the additional data to the data-clustering ML model, and (ii) the plurality of ML models generating the personalized communication is further based on the data clusters.

### Further Consideration

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects.

Thus, the claims are not intended to be limited to the aspects shown herein but are to be accorded the full scope consistent with the language of the claims. For example, reference to an element in the singular is not intended to mean “one and only one” unless specifically so stated, but rather “one or more.” Moreover, unless specifically stated otherwise, the term “some” refers to one or more. Pronouns in the masculine (e.g., his) include the feminine and neuter genders (e.g., her and its) and vice versa. Headings and subheadings, if any, are used for convenience only and do not limit the invention described herein.

What is claimed is:

1. A system for improving adherence to patient treatment plans, the system comprising:
a transformer configured to vectorize data regarding a patient;
a storage layer configured to store the data;
a plurality of machine learning (ML) models configured to generate a personalized communication for the patient based on the data, wherein the plurality of ML models comprises (i) a plan-complexity ML model trained using historical adherence data associated with treatment plans similar to a treatment plan prescribed for the patient, (ii) a patient-persona ML model trained using behavioral pattern data associated with patients similar to the patient, (iii) an adherence-probability ML model trained using the historical adherence data and persona data associated with patients similar to the patient, and (iv) a personalized-communication ML model trained using interaction data comprising language from interactions involving other patients similar to the patient;
a delivery network configured to deliver the personalized communication to the patient; and
an electronic device comprising a processor configured to:
receive the data regarding the patient, wherein the data comprises (i) treatment plan data regarding the treatment plan prescribed for the patient, and (ii) other data regarding a healthcare provider associated with the patient, a clinician associated with the patient, a medical record associated with the patient, a social media account associated with the patient, a laboratory associated with the patient, or a pharmacy associated with the patient;
vectorize the data using the transformer, wherein vectorizing the data comprises translating the treatment plan data and the other data into numerical representations that capture semantic meaning embedded within the treatment plan data and the other data;
store the data in the storage layer after vectorizing the data;
provide the treatment plan data from the storage layer to the plan-complexity ML model, wherein the treatment plan data comprises (i) a number of medications prescribed in the treatment plan, and (ii) a dosage frequency for the prescribed medications;
receive a plan-complexity score from the plan-complexity ML model responsive to providing the treatment plan data to the plan-complexity ML model, wherein the plan-complexity score indicates a complexity level of the treatment plan;
provide the other data from the storage layer to the patient-persona ML model, wherein the other data regards at least (i) the medical record associated with the patient and (ii) the social media account associated with the patient;
receive persona indicators from the patient-persona ML model responsive to providing the other data to the patient-persona ML model, wherein the persona indicators indicate (i) an ability of the patient to follow a prescribed regime and (ii) a communication style of the patient;
provide the treatment plan data from the storage layer, the plan-complexity score, and the persona indicators to the adherence-probability ML model;
receive an adherence-probability score from the adherence-probability ML model responsive to providing the treatment plan data, the plan-complexity score, and the persona indicators to the adherence-probability ML model, wherein the adherence-probability score indicates a probability of the patient adhering to the treatment plan;
provide the treatment plan data from the storage layer, the adherence-probability score, and the persona indicators to the personalized-communication ML model;
receive a communication plan and the personalized communication from the personalized-communication ML model, wherein (i) the communication plan indicates whether the personalized communication should include text, images, or video and whether the personalized communication should be delivered via email, text message, notification, phone call, or letter and (ii) the personalized communication is addressed to the patient and regards the treatment plan prescribed for the patient; and
deliver the personalized communication to the patient via the delivery network and according to the communication plan.

2. An electronic device configured to generate personalized communications regarding patient treatment plans, the electronic device comprising a processor configured to:
receive data regarding a patient, wherein the data comprises (i) treatment plan data regarding a treatment plan prescribed for the patient and (ii) other data regarding a healthcare provider associated with the patient, a clinician associated with the patient, a medical record associated with the patient, a social media account associated with the patient, a laboratory associated with the patient, or a pharmacy associated with the patient;
provide the data to a plurality of ML models configured to generate a personalized communication for the patient based on the data;
receive the personalized communication from the plurality of ML models responsive to providing the data to the plurality of ML models, wherein the personalized communication is addressed to the patient and regards the treatment plan prescribed for the patient; and
deliver the personalized communication to the patient.

3. The electronic device of claim 2, wherein the plurality of ML models comprises a plan-complexity ML model configured to:
receive the treatment plan data, wherein the treatment plan data comprises three or more of (i) a number of medications prescribed in the treatment plan, (ii) an availability of the prescribed medications, (iii) a dosage frequency for the prescribed medications, (iv) a number of chronic conditions addressed by the treatment plan, (v) an indication that one or more of the prescribed medications is a high-risk medication, (vi) a dietary restriction prescribed in the treatment plan, and (vii) an indication that the prescribed treatment plan requires special medical equipment; and
generate a plan-complexity score based on the treatment plan data, wherein the plan-complexity score indicates a complexity level of the treatment plan;
wherein (i) providing the data to the plurality of ML models comprises providing the treatment plan data to the plan-complexity ML model and (ii) the plurality of ML models generating the personalized communication is further based on the treatment-plan-complexity score.

4. The electronic device of claim 3, wherein the plurality of ML models comprises a patient-persona ML model configured to:
receive the other data, wherein the other data regards at least (i) the medical record associated with the patient and (ii) the social media account associated with the patient; and
generate persona indicators based on the other data, wherein the persona indicators indicate two or more of (i) an organization level of the patient, (ii) an ability of the patient to follow a prescribed regime, and (iii) a communication style of the patient;
wherein (i) providing the data to the plurality of ML models comprises providing the other data to the patient-persona ML model and (ii) the plurality of ML models generating the personalized communication is further based on the persona indicators.

5. The electronic device of claim 4, wherein the plurality of ML models further comprises an adherence-probability ML model configured to:
receive the treatment plan data;
receive the plan-complexity score from the plan-complexity ML model;
receive the persona indicators from the patient-persona ML model; and
generate an adherence-probability score for the patient based on the treatment plan data, the plan-complexity score, and the persona indicators, wherein the adherence-probability score indicates a probability of the patient adhering to the treatment plan;
wherein (i) providing the data to the plurality of ML models comprises providing the treatment plan data to the adherence-probability ML model, (ii) the plan-complexity ML model is further configured to provide the plan-complexity score to the adherence-probability ML model, and (iii) the patient-persona ML model is further configured to provide the persona indicators to the adherence probability ML model, and (iv) the plurality of ML models generating the personalized communication is further based on the adherence-probability score.

6. The electronic device of claim 5, further comprising a feedback channel configured to:
receive feedback from the patient regarding the personalized communication, wherein the feedback indicates two or more of (i) an amount of the personalized communication reviewed by the patient, (ii) a response of the patient to the personalized communication, and (iii) an affect of the personalized communication on adherence to the treatment plan by the patient; and
provide the feedback to (i) the clinician and (ii) the adherence-probability ML model, wherein the adherence-probability ML model is further configured to receive the feedback, and wherein the adherence-probability ML model generating the adherence-probability score is further based on the feedback.

7. The electronic device of claim 5, wherein the plurality of ML models further comprises a personalized-communication ML model configured to:
receive the treatment plan data;
receive the adherence-probability score from the adherence-probability ML model;
receive the persona indicators from the patient-persona ML model; and
generate the personalized communication based on the treatment plan data, the adherence-probability score, and the persona indicators;
wherein (i) providing the data to the plurality of ML models comprises providing the treatment plan data to the personalized-communication ML model, (ii) the adherence-probability ML model is further configured to provide the adherence-probability score to the personalized-communication ML model, and (iii) the patient-persona ML model is further configured to provide the persona indicators to the personalized-communication ML model.

8. The electronic device of claim 7, wherein the personalized-communication ML model is further configured to:
prior to generating the personalized communication, generate a communication plan based on the treatment plan data, the adherence-probability score, and the persona indicators, wherein the communication plan indicates (i) whether the personalized communication should include text, images, or video and (ii) whether the personalized communication should be delivered via email, text message, notification, phone call, or letter;
wherein the personalized-communication ML model generating the personalized communication is further based on the communication plan, and delivering the personalized communication to the patient comprises delivering the personalized communication according to the communication plan.

9. The electronic device of claim 2, further comprising:
a storage layer configured to (i) store the data regarding the patient and (ii) store additional data regarding additional patients; and
a search engine configured to (i) receive a portion of the data as a query and (ii) locate a relevant portion of the additional data that is similar to the query;
wherein the electronic device is further configured to (i) store the data in the storage layer, (ii) receive the additional data, (iii) store the additional data in the storage layer, (iv) determine that the data should be supplemented with the additional data, and (v) responsive to determining that the data should be supplemented, retrieve the relevant portion of the additional data using the search engine and provide the relevant portion of the additional data to the plurality of ML models prior to receiving the personalized communication; and
wherein the plurality of ML models generating the personalized communication is further based on the relevant portion of the additional data.

10. The electronic device of claim 9, wherein the plurality of ML models comprises a data-clustering ML model configured to:
receive the data and the relevant portion of the additional data; and
generate data clusters of the data and the relevant portion of the additional data, wherein the data clusters are grouped according to data type and risk of non-adherence;
wherein (i) providing the data to the plurality of ML models comprises providing the data to the data-clustering ML model, (ii) providing the relevant portion of the additional data to the plurality of ML models comprises providing the relevant portion of the additional data to the data-clustering ML model, and (ii) the plurality of ML models generating the personalized communication is further based on the data clusters.

11. The electronic device of claim 9, wherein the storage layer comprises:
an object data store configured to store the data regarding the patient and the additional data regarding the additional patients;
a transformer configured to vectorize the data and the additional data; and
a vector database configured to store the data and the additional data after the transformer vectorizes the data and the additional data;
wherein the query comprises a vectorized portion of the data, and the search engine retrieving the relevant portion of the additional data comprises the search engine retrieving the relevant portion of the additional data from the vector database.

12. A computer-implemented method for improving adherence to patient treatment plans, the method comprising:
receiving data regarding a patient, wherein the data comprises (i) treatment plan data regarding a treatment plan prescribed for the patient, and (ii) other data regarding a healthcare provider associated with the patient, a clinician associated with the patient, a medical record associated with the patient, a social media account associated with the patient, a laboratory associated with the patient, or a pharmacy associated with the patient;
providing the data to a plurality of ML models configured to generate a personalized communication for the patient based on the data;
generating the personalized communication using the plurality of ML models after providing the data to the plurality of ML models, wherein the personalized communication is addressed to the patient and regards the treatment plan prescribed for the patient;
receiving the personalized communication from the plurality of ML models after generating the personalized communication; and
delivering the personalized communication to the patient.

13. The computer-implemented method of claim 12, wherein the plurality of ML models comprises a plan-complexity ML model configured to:
receive the treatment plan data, wherein the treatment plan data comprises three or more of (i) a number of medications prescribed in the treatment plan, (ii) an availability of the prescribed medications, (iii) a dosage frequency for the prescribed medications, (iv) a number of chronic conditions addressed by the treatment plan, (v) an indication that one or more of the prescribed medications is a high-risk medication, (vi) a dietary restriction prescribed in the treatment plan, and (vii) an indication that the prescribed treatment plan requires special medical equipment; and
generate a plan-complexity score based on the treatment plan data, wherein the plan-complexity score indicates a complexity level of the treatment plan;
wherein (i) providing the data to the plurality of ML models comprises providing the treatment plan data to the plan-complexity ML model and (ii) the plurality of ML models generating the personalized communication is further based on the treatment-plan-complexity score.

14. The computer-implemented method of claim 13, wherein the plurality of ML models comprises a patient-persona ML model configured to:
receive the other data, wherein the other data regards at least (i) the medical record associated with the patient and (ii) the social media account associated with the patient; and
generate persona indicators based on the other data, wherein the persona indicators indicate (i) an organization level of the patient, (ii) an ability of the patient to follow a prescribed regime, and (iii) a communication style of the patient;
wherein (i) providing the data to the plurality of ML models comprises providing the other data to the patient-persona ML model and (ii) the plurality of ML models generating the personalized communication is further based on the persona indicators.

15. The computer-implemented method of claim 14, wherein the plurality of ML models further comprises an adherence-probability ML model configured to:
receive the treatment plan data;
receive the plan-complexity score from the plan-complexity ML model;
receive the persona indicators from the patient-persona ML model; and
generate an adherence-probability score for the patient based on the treatment plan data, the plan-complexity score, and the persona indicators, wherein the adherence-probability score indicates a probability of the patient adhering to the treatment plan;
wherein (i) providing the data to the plurality of ML models comprises providing the treatment plan data to the adherence-probability ML model, (ii) the plan-complexity ML model is further configured to provide the plan-complexity score to the adherence-probability ML model, and (iii) the patient-persona ML model is further configured to provide the persona indicators to the adherence probability ML model, and (iv) the plurality of ML models generating the personalized communication is further based on the adherence-probability score.

16. The computer-implemented method of claim 15, further comprising:
receiving feedback from the patient regarding the personalized communication, wherein the feedback indicates two or more of (i) an amount of the personalized communication reviewed by the patient, (ii) a response of the patient to the personalized communication, and (iii) an affect of the personalized communication on adherence to the treatment plan by the patient; and
providing the feedback to (i) the clinician and (ii) the adherence-probability ML model, wherein the adherence-probability ML model is further configured to receive the feedback, and wherein the adherence-probability ML model generating the adherence-probability score is further based on the feedback.

17. The computer-implemented method of claim 15, wherein the plurality of ML models further comprises a personalized-communication ML model configured to:
receive the treatment plan data;
receive the adherence-probability score from the adherence-probability ML model;
receive the persona indicators from the patient-persona ML model; and
generate the personalized communication based on the treatment plan data, the adherence-probability score, and the persona indicators;
wherein (i) providing the data to the plurality of ML models comprises providing the treatment plan data to the personalized-communication ML model, (ii) the adherence-probability ML model is further configured to provide the adherence-probability score to the personalized-communication ML model, and (iii) the patient-persona ML model is further configured to provide the persona indicators to the personalized-communication ML model.

18. The computer-implemented method of claim 17, wherein the personalized-communication ML model is further configured to:
prior to generating the personalized communication, generate a communication plan based on the treatment plan data, the adherence-probability score, and the persona indicators, wherein the communication plan indicates (i) whether the personalized communication should include text, images, or video and (ii) whether the personalized communication should be delivered via email, text message, notification, phone call, or letter;
wherein the personalized-communication ML model generating the personalized communication is further based on the communication plan, and delivering the personalized communication to the patient comprises delivering the personalized communication according to the communication plan.

19. The computer-implemented method of claim 12, further comprising:
storing the data regarding the patient in a storage layer;
receiving additional data regarding additional patients;
storing the additional data regarding the additional patients in the storage layer;
determining that the data should be supplemented with the additional data;
responsive to determining that the data should be supplemented, (i) retrieving a relevant portion of the additional data using a search engine and (ii) providing the relevant portion of the additional data to the plurality of ML models prior to receiving the personalized communication;
wherein the search engine is configured to receive a portion of the data as a query and locate the relevant portion of the additional data that is similar to the query, and the plurality of ML models generating the personalized communication is further based on the relevant portion of the additional data.

20. The computer-implemented method of claim 19, wherein the plurality of ML models comprises a data-clustering ML model configured to:
receive the data and the relevant portion of the additional data; and
generate data clusters of the data and the relevant portion of the additional data, wherein the data clusters are grouped according to data type and risk of non-adherence;
wherein (i) providing the data to the plurality of ML models comprises providing the data to the data-clustering ML model, (ii) providing the relevant portion of the additional data to the plurality of ML models comprises providing the relevant portion of the additional data to the data-clustering ML model, and (ii) the plurality of ML models generating the personalized communication is further based on the data clusters.
